(19) United States
(12) Patent Application Publication
(10) Pub. No.: US 2019/0293540 A1
(43) Pub. Date: Sep. 26, 2019

(54) Anti-Biofouling Graphene Coated Micro Sensors and Methods for Fabricating the Same

(71) Applicant: SPAWAR Systems Center Pacific, San Diego, CA (US)

(72) Inventors: Jonathon K. Oiler, Fort Collins, CO (US); Mitchell B. Lerner, San Diego, CA (US)

(73) Assignee: United States of America as represented by Secretary of the Navy, San Diego, CA (US)

(21) Appl. No.: 15/927,833

(22) Filed: Mar. 21, 2018

(51) Int. Cl. G01N 15/06; G01N 33/18; C09D 1/00; C09D 5/16; C09D 5/08

(52) U.S. Cl. CPC G01N 15/0656; G01N 33/1886; C09D 1/00; G01N 2015/0053; C09D 5/08; C09D 5/1693; C09D 5/1656

(57) ABSTRACT

A sensing device includes a plurality of micro sensors configured to detect electrical conductivity. The micro sensors are coated with graphene. The graphene prevents biofouling of the micro sensors.

### FEDERALLY-SPONSORED RESEARCH AND DEVELOPMENT

The United States Government has ownership rights in this invention. Licensing inquiries may be directed to Office of Research and Technical Applications, Space and Naval Warfare Systems Center, Pacific, Code 72120, San Diego, Calif., 92152; telephone (619) 553-5118; email: ssc_pac_t2@navy.mil, referencing NC 103,007.

### FIELD OF THE INVENTION

The present invention pertains generally to graphene coated structures. More particularly, the present invention pertains to anti-biofouling graphene coated micro sensors.

### BACKGROUND OF THE INVENTION

Measurement of seawater salinity is important for many applications. For example, salinity, which is the measure of the concentration of salts in water, plays an important role in determining the acoustic velocity in seawater.

Acoustic velocity (or sound speed) in water is a function of the density of the water which is affected by the water temperature, salinity, and pressure. Acoustic velocity is a key parameter for determining the location of an object underwater when using sonar. Surface vessels and submarines rely on in-situ salinity data to provide values for sonar measurements. Thus, it is important that sensors for determining acoustic velocity be protected from biofouling and corrosion.

In-situ salinity is determined by using a proxy measurement of the water's electrical conductivity. The electrical conductivity of the water is a measure of the number of ions per unit volume of water. Since the vast majority of ions in seawater are due to salts, the electrical conductivity measurement provides a suitable method for measuring the seawater salinity.

Electrical conductivity of seawater is typically measured by passing a known current through the seawater between two electrodes and measuring the voltage drop across the seawater through which the current passes. According to Ohm's Law, the resistance of the seawater can be obtained by dividing the measured voltage drop by the known current. The inverse of the resistance is conductance, the value of which is used in the determination of the salinity.

Because electrical conductivity is really a measurement of resistance, the electrical path of the current from one electrode to the other electrode and the electrical resistance at the interface between the exposed electrodes and the seawater must be well characterized and taken into account at sensor calibration. Biofilms that grow on the surface of the electrodes after calibration will change the impedance at the electrode-seawater interface, resulting in the loss of sensitivity of the sensor and causing electronic drift.

Biofilms are groups of microorganisms that grow on the electrode surface during exposure to seawater and are commonly referred to as “biofoulants”. The process of the microorganisms attaching to a surface is commonly referred to as “biofouling”. Once a sensor has undergone biofouling, the data output by the sensor is no longer reliable. Therefore, biofouling significantly decreases the lifetime of the sensor.

Conventionally, elaborate and expensive techniques have been used to minimize biofouling for conductivity sensors. One technique involves pumping seawater away from the electrodes during times of sensor inactivity to limit the exposure of seawater and thus biofoulants to the electrodes.

Another technique employs a micro-pump and Tributyltin. Tributyltin is a biocidal agent that is toxic to microorganisms. The pump periodically washes the electrode surface with the biocide. This process requires a limited reservoir of the chemical which is dangerous in larger concentrations to aquatic and human life.

Additionally, copper meshes or coatings are sometimes employed around a conductivity sensor. The copper ions also can act as a biocide to organisms that may attach to the electrodes. Hydrophobic coatings are also employed as biocides. However, copper and hydrophobic coatings have not proven to be suitably effective and may be toxic.

While all of these techniques prevent biofouling to some extent, they are costly and/or complex to employ.

In view of the above, it would be desirable to provide a simple, inexpensive sensing device that is protected from biofoulants and corrosion.

### SUMMARY OF THE INVENTION

According to an illustrative embodiment, a sensing device includes a plurality of micro sensors configured to detect electrical conductivity. The micro sensors are coated with graphene. The graphene prevents biofouling of the micro sensors.

These, as well as other objects, features and benefits will now become clear from a review of the following detailed description, the illustrative embodiments, and the accompanying drawings.

### BRIEF DESCRIPTION OF THE DRAWINGS

The novel features of the present invention will be best understood from the accompanying drawings, taken in conjunction with the accompanying description, in which similarly-referenced characters refer to similarly-referenced parts, and in which:

FIG. 1A illustrates biofouling of a surface;

FIG. 1B illustrates prevention of biofouling of a surface according to an illustrative embodiment.

FIG. 2 illustrates a cross-sectional view of a sensing device in an aqueous solution according to illustrative embodiments.

FIG. 3 depicts a top view of electrodes included in a sensing device according to an illustrative embodiment.

FIG. 4 is a flow chart showing steps in a process for fabricating a sensing device according to one embodiment.

FIG. 5 is a flow chart showing steps in a process for fabricating a sensing device according to another embodiment.

### DETAILED DESCRIPTION OF THE EMBODIMENTS

According to illustrative embodiments, a sensing device including micro sensors is fabricated with a graphene layer covering the micro sensors. The graphene layer is selectively applied to the top surfaces of the micro sensors, preventing biofouling of the micro sensors when placed in an aqueous solution, such as seawater. The graphene layer also prevents corrosion of the micro sensors.

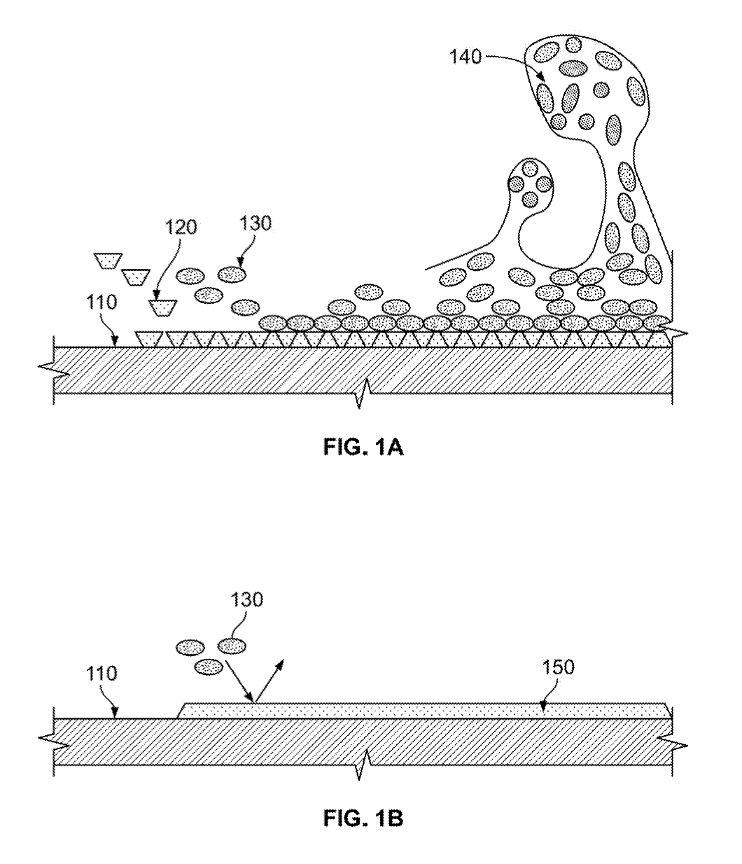

As an aid to understanding illustrative embodiments, the process of biofouling is described with reference to FIG. 1A. As shown in FIG. 1A, the biofouling process begins with a substrate 110 being placed into an aqueous environment prone to biofouling, such as seawater. The substrate 110 absorbs a conditioning film 120 made of polymer seeds. The conditioning film 120 coats the surface of the substrate 110. Next, free-floating bacterium 130 contact and are absorbed onto the coated substrate 110, using the conditioning film 120 as foodstuff. As more bacterium 130 are absorbed, a biofilm is produced including an extracellular polymer matrix 140 of embedded micro colonies. The result is biofouling of the substrate surface.

FIG. 1B illustrates how a graphene layer on a surface prevents biofouling according to an illustrative embodiment. Graphene is a flexible and robust material that can conform to almost any surface and bind strongly. Also, graphene has intrinsic hydrophobic properties.

As shown in FIG. 1B, when the substrate 110 that is placed into the aqueous solution is coated with graphene 150, the intrinsic hydrophobic properties of the graphene 150 cause the bacterium 130 to be repelled from the surface of the substrate 110. This prevents corrosion of the surface of the substrate 110. In addition, in an aqueous solution such as seawater that is particularly prone to biofouling, coating the substrate with the graphene 150 prevents absorption of polymer seeds, thus preventing coating of the substrate by the conditioning film 120. Prevention of coating with the conditioning film 120 prevents absorption of bacterium 130, thus preventing biofouling of the substrate surface.

According to illustrative embodiments, the surfaces of micro sensors are coated with graphene to prevent biofouling and corrosion of the micro sensors. This may be understood with reference to FIG. 2 which illustrates a cross-sectional view of a sensing device in water according to illustrative embodiments.

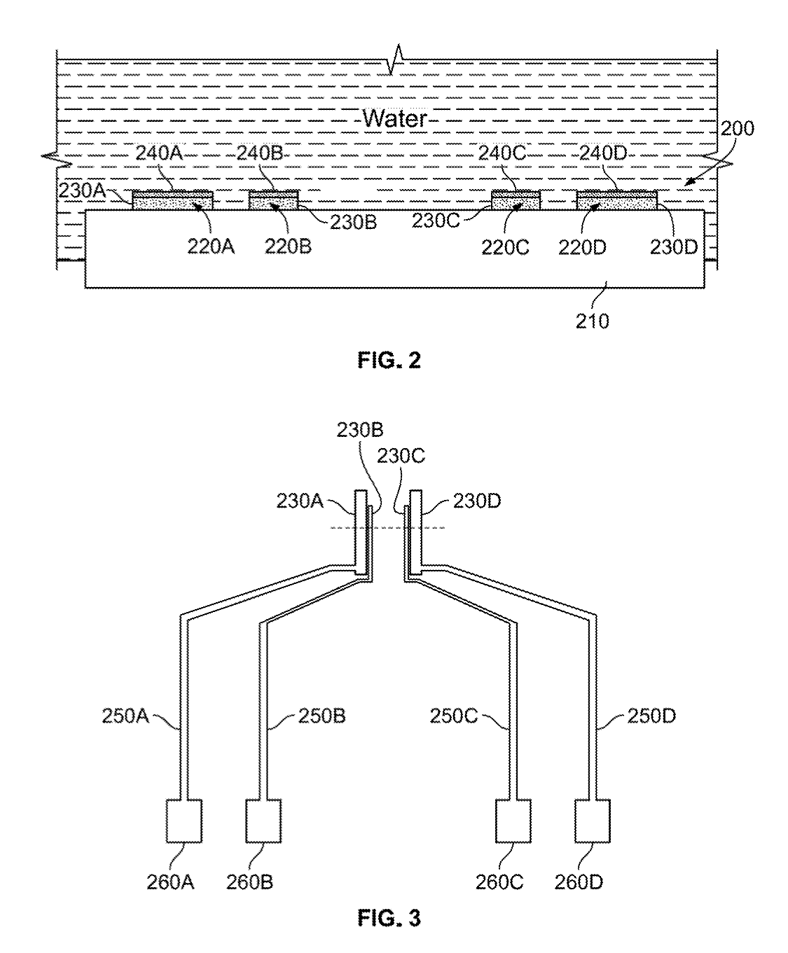

Referring to FIG. 2, the sensing device 200 includes a plurality of micro sensors 220A, 220B, 220C, and 220D on a substrate 210. The substrate 210 includes an insulating material. The outer micro sensors 220A and 220D are used to pass current through seawater, and the inner micro sensors 220B and 220C are used to measure a voltage drop as described in more detail with reference to FIG. 3.

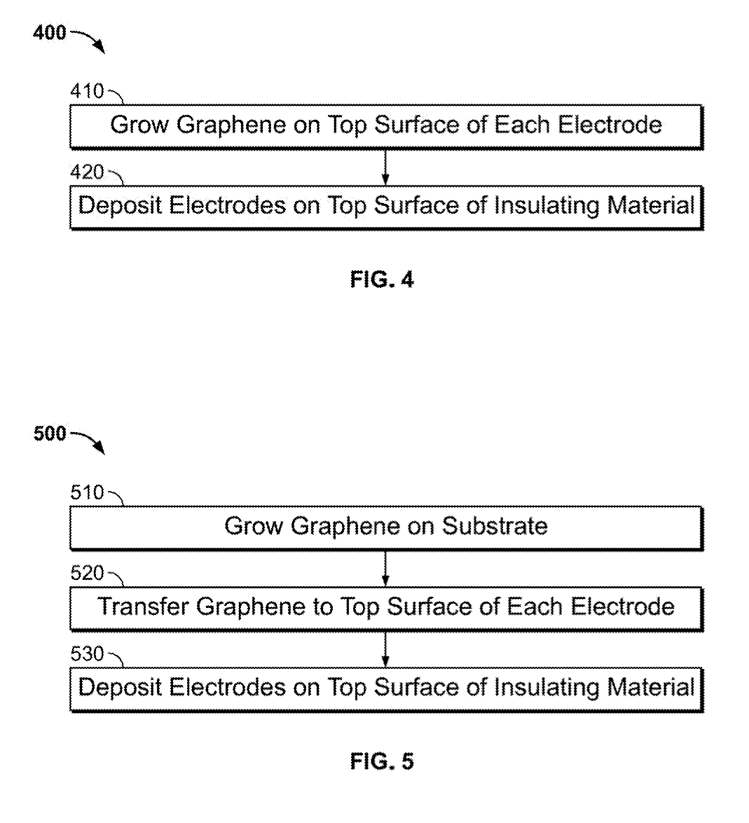

Each micro sensor includes, for example, an electrode coated on a top surface with graphene. That is, the micro sensor 220A includes an electrode 230A coated with a graphene layer 240A, the micro sensor 220B includes an electrode 230B coated with a graphene layer 240B, the micro sensor 220C includes an electrode coated with a graphene layer 240C, and the micro sensor 220D includes an electrode 230D coated with a graphene layer 240D. Each electrode may be coated with graphene in various manners, described in detail below with reference to FIGS. 4 and 5. Coating of the top surface of each electrode with the graphene prevents biofouling and corrosion of the electrodes.

Because the graphene can be selectively deposited on the electrode surfaces, the electrodes can be specifically targeted for anti-biofouling and anti-corrosion. This allows the size of the sensing device to be minimized as no meshes or pumps are necessary. While the graphene adds some resistance, this may be characterized and accounted for with calibration.

It should be appreciated that FIG. 2 is not to scale. That is, the electrodes 230A, 230B, 230C and 230D may be approximately 100 nanometers (nm) thick, while the graphene layers 240A, 240B, 240C and 240D may be the thickness of a single atomic layer.

FIG. 3 depicts a top view of electrodes included in the sensing device shown in FIG. 2. The substrate and graphene are not depicted in FIG. 3 for ease of illustration.

Referring to FIG. 3, electrodes 230A, 230B, 230C, and 230D are respectively connected by leads 250A, 250B, 250C and 250D to bond pads 260A, 260B, 260C and 260D. The electrodes 230A, 230B, 230C, and 230D may be made of any suitable metal, such as platinum. The leads 250A, 250B, 250C and 250D and the bond pads 260A, 260B, 260C and 260D may be made of any suitable electrical conductive material. The bond pads 260A, 260B, 260C and 260D, are, in turn, connected to signal-conditioning circuitry which provides a known electrical current and receives a detected signal as described in more detail below. The signal conditioning circuitry is not shown for ease of illustration.

The electrodes 230A, 230B, 230C, and 230D are configured to detect electrical conductivity in water. In the arrangement shown in FIG. 3, the outer two electrodes 230A and 230D are used to pass a known electrical current received from a current source (not shown), connected to the bond pads 260A and 260D via lead lines 250A and 250D, through seawater. The current is passed on paths from the electrodes 230A and 230D towards the electrodes 230B and 230C, respectively. In turn, the inner two electrodes 230B and 230C are used to detect the voltage drop through the seawater on the paths from the electrodes 230A and 230D, respectively. This voltage signal is passed via lead lines 250B and 250C to bond pads 260B and 260C to be used by signal processing circuity (not shown) for determining the resistance and electrical conductivity of the seawater. Based on the electrical conductivity, the salinity of the seawater may be determined in a manner those of ordinary skill in the art would understand.

It should be appreciated that, while FIG. 2 illustrates a sensing device including four electrodes, referred to as a “four point probe”, and FIG. 3 depicts an arrangement of four electrodes, a sensing device may be implemented with any number of electrodes. For example, a three point probe may be implemented with three electrodes, while a two point probe may be implemented with two electrodes. Graphene coating as described herein may be used for any configuration of sensors that measures electrical conductivity and exposes the electrodes to seawater or another aqueous solution prone to biofouling.

A sensing device covered with graphene as described above allows for smaller, lower cost and longer lifetime sensors. In addition, the sensing device will use less power overall than one that would require a pump.

While the sensing device described above includes electrodes coated with graphene, other conducting nanomaterials, such as carbon nanotubes, silicon, or graphene oxide may be used instead of graphene, as these conducting nanomaterials have similar anti-corrosion and anti-biofouling properties. For a very thin layer, the added resistance through these other materials may be minimal.

The sensing device described above may be fabricated using various techniques. According to one embodiment, the sensing device is fabricated by growing a graphene layer directly on the top surface of each electrode and then attaching the bottom surface of each electrode to an insulating substrate. This technique is described in detail below with reference to FIG. 4.

FIG. 4 is a flow chart showing steps in a process for fabricating a sensing device according to one embodiment. The process 400 begins at step 410 at which graphene is selectively grown directly on the top surface of each of the electrodes, e.g., the electrodes 230A, 230B, 230C and 230D shown in FIGS. 2 and 3. The graphene may be grown directly on the electrodes by, for example, chemical vapor deposition.

At step 420, the electrodes are deposited on a top surface of an insulating material, such as the substrate 210, such that a bottom surface of each of the electrodes contacts the top surface of the substrate. The electrodes may be deposited on the insulating material in any suitable manner.

Growing the graphene directly on the electrodes provides for intimate contact between the graphene and the electrodes, thus reducing the possibility of seawater reaching the metal electrodes. However, this technique requires that the micro sensor be robust enough to be placed in a furnace at high temperature in the presence of a carbon-containing gas for the production of graphene.

According to another embodiment, the sensing device may be fabricated by growing graphene on a substrate, transferring the graphene to the top surface of each electrode, and then attaching a bottom surface of each electrode to an insulating substrate. This technique is described in detail below with reference to FIG. 5.

FIG. 5 is a flow chart showing steps in a process for fabricating a sensing device according to another embodiment. The process 500 begins at step 510 at which graphene is grown as a single layer or multiple layers on a substrate via a chemical vapor deposition process, a mechanical exfoliation process, an epitaxial growth process, or any other suitable process.

At step 520, the graphene is transferred to the top surface of each of the electrodes, e.g., the electrodes 230A, 230B, 230C and 230D shown in FIGS. 2 and 3. This may include removing the graphene from the substrate on which it is grown. This removal may be performed electrochemically, chemically, with thermal release tape, or any other suitable method. For example, In the case of graphene being grown on a copper foil by chemical vapor deposition, the graphene can be removed from the copper foil by bubble transfer, chemical etching or any other method.

In the case of bubble transfer, the graphene may be supported by a polymethyl methacrylate (PMMA) layer. The graphene is grown at high temperatures, e.g., approximately 1050 degrees Celsius.

The graphene can be removed from the copper foil by bubble transfer or chemical etching. In the case of bubble transfer, the graphene layer, supported by a PMMA layer, is electrochemically separated from the copper by using electrodes to apply a voltage between the copper sheet and a bath containing NaOH. Bubbles form at the electrodes, lifting off the graphene/PMMA stack. Similarly, the PMMA/graphene/copper could be placed in an etchant, such as iron chloride or ammonium persulfate to etch away the copper, thus leaving the PMMA/graphene layers. When the PMMA/graphene is separated from the copper foil, the graphene/PMMA stack can be transferred to the top surfaces of the electrodes, e.g., the electrodes 230A, 230B, 230C and 230D shown in FIGS. 2 and 3.

For chemical etching, the PMMA/graphene/copper could be placed in an etchant, such as iron chloride or ammonium persulfate to etch away the copper, thus leaving the PMMA/graphene layers. The PMMA/graphene can then be transferred to the top surfaces of the electrodes, e.g., the electrodes 230A, 230B, 230C and 230D shown in FIGS. 2 and 3. The PMMA can then be washed away in acetone and the sample is annealed in hydrogen/argon environment at 200° C. to finish the cleaning process.

Referring again to FIG. 5, the process 500 ends by depositing the electrodes on the top surface of an insulating material, such as the substrate 210 in FIG. 2.

It should be appreciated that the steps and order of steps described and illustrated are provided as examples. Fewer, additional, or alternative steps may also be involved and/or some steps may occur in a different order.

In both the techniques described above with reference to FIGS. 4 and 5, the electrodes may be deposited on the substrate in any manner such that the electrodes are adhered to the substrate. It is expected that the anti-biofouling properties of the graphene would be independent of the any anti-biofouling properties that the substrate may have (or not have).

In addition, for both techniques described above, the graphene material may be characterized by Raman spectroscopy or DC electrical measurements to ensure high quality, e.g., before being applied to the surface of each electrode. Further, the graphene may be chemically treated to create functionalized graphene, e.g., to increase the hydrophobicity of the graphene or to include some biocidal properties.

The sensing devices described herein may be used for various applications, such as determining acoustic velocity.

Also, the techniques described herein may be used to minimize biofouling and corrosion on surfaces other than electrical conductivity sensors, such as ship hulls. Biofouling creates drag for a ship, resulting in increased fuel usage. This increase in fuel usage results in increased costs and adverse environmental effects due to carbon dioxide and sulfur dioxide emissions.

Further, the issue of biofouling extends beyond the shipping industry to any application where a surface is exposed to water. Other affected industries include, for example, water purification (such as reverse osmosis systems), industrial cooling for large equipment or power stations, oil pipelines, drug delivery systems, papermaking machines, fire sprinkler delivery systems, and underwater instruments. In addition to the increased fuel costs due to biofouling, there is potential for instrumentation failure and the added cost of replacing damaged components. A passive anti-biofouling coating that can be made inexpensively in large quantities may be significant for reducing these effects.

Thus, the techniques described above may be used in any situation that requires a passive anti-biofouling coating solution. Maritime assets could benefit from such a technology, as could water purification systems (such as reverse osmosis systems), industrial cooling for large equipment or power stations, oil pipelines, drug delivery systems, papermaking machines, fire sprinkler delivery systems, other underwater instruments and any industry that involves water contacting a surface.

By integrating the hydrophobicity, scalability, and adhesiveness of a graphene coating, the resulting anti-biofouling coating solution can be expected to perform better than tin-based coating solutions which are toxic to marine organisms. With improvements in the manufacturing scale of graphene, it may possible to coat a large surface, such as an entire ship, to prevent biofouling and corrosion indefinitely.

It will be understood that many additional changes in the details, materials, steps and arrangement of parts, which have been herein described and illustrated to explain the nature of the invention, may be made by those skilled in the art within the principle and scope of the invention as expressed in the appended claims.

What is claimed is:

1. A sensing device, comprising:
a plurality of micro sensors configured to detect electrical conductivity; and
a graphene layer coating the micro sensors, wherein the graphene layer prevents biofouling of the micro sensors.

2. The sensing device of claim 1, wherein the graphene layer also prevents corrosion of the micro sensors.

3. The sensing device of claim 1, wherein the graphene layer is grown directly on the micro sensors.

4. The sensing device of claim 1, wherein the graphene layer is grown separately from the micro sensors and transferred to a surface of each of the micro sensors.

5. The sensing device of claim 1, wherein the micro sensors include electrodes deposited on an insulating material.

6. The sensing device of claim 5, wherein the graphene layer is applied to a surface of each of the electrodes.

7. The sensing device of claim 1, wherein the micro sensors are configured to detect electrical conductivity in water.

8. The sensing device of claim 7, wherein the detected electrical conductivity in water represents salinity of the water.

9. A method for fabricating a sensing device, comprising:
growing a graphene layer directly on a top surface of each of a plurality of electrodes configured for sensing electrical conductivity, wherein the graphene layer prevents biofouling and corrosion of the electrodes; and
depositing the electrodes, with the graphene grown on the top surface of each electrode, on a top surface of an insulating material, such that a bottom surface of each of the electrodes contacts the top surface of the insulating material.

10. The method of claim 9, wherein the graphene layer is grown on the top surface of each of the electrodes by chemical vapor deposition.

11. The method of claim 9, wherein the graphene layer is grown on the top surface of each of the electrodes by placing the electrodes in a furnace in a presence of a carbon-containing gas, such that the graphene layer forms on the top surface of each of the electrodes.

12. A method for fabricating a sensing device, comprising:
growing at least one graphene layer;
transferring the graphene layer to a top surface of each of a plurality of electrodes configured for sensing electrical conductivity, wherein the graphene layer prevents biofouling and corrosion of the electrodes;
depositing the electrodes on a top surface of an insulator material, such that a bottom surface of each of the electrodes contacts the top surface of the insulator material.

13. The method of claim 12, wherein multiple layers of graphene are grown and transferred to the top surface of each of the electrodes.

14. The method of claim 13, wherein the graphene layer is grown on a substrate.

15. The method of claim 14, further comprising removing the graphene layer from the substrate for transfer to the electrodes.

16. The method of claim 13, wherein the graphene layer is grown by chemical vapor deposition on copper foil.

17. The method of claim 16, further comprising removing the graphene layer from the copper foil by at least one of chemical etching and bubble transfer.

18. The method of claim 13, wherein the graphene layer is grown by mechanical exfoliation.

19. The method of claim 13, wherein the graphene layer is grown epitaxially.

20. The method of claim 13, wherein the graphene layer is grown by chemical synthesis.
